(19) United States
(12) Patent Application Publication
(10) Pub. No.: US 2026/0248606 A1
(43) Pub. Date: Aug. 27, 2026

(54) PRESERVED CORNEAL PLUGS AND ASSOCIATED SYSTEMS, DEVICES AND METHODS FOR FABRICATING CORNEAL PLUGS

(71) Applicant: University of Iowa Research Foundation, lowa City, IA (US)

(72) Inventor: Christopher Sales, Coralville, IA (US)

(73) Assignee: University of Iowa Research Foundation, lowa City, IA (US)

(21) Appl. No.: 18/872,698

(22) PCT Filed: Jun. 8, 2023
(86) PCT No.: PCT/US2023/024839

§ 371 Date: Dec. 6, 2024

Related U.S. Application Data
(60) Provisional application No. 63/350,157, filed on Jun. 8, 2022.

(51) Int. Cl. A61F 2/14; A61F 2/00; A61L 27/36

(52) U.S. Cl. CPC A61F 2/142; A61L 27/3604; A61F 2002/0081; A61F 2240/001; A61L 2430/16

(57) ABSTRACT

A corneal plug comprising preserved human corneal tissue with an encircling groove along the perimeter of the tissue. A method for creating a corneal plug comprising punching a plug from a corneal allograft that has been preserved, trimming the plug to a selected thickness with a cutting device, and cutting an encircling groove around the plug. A device for creating a corneal plug comprising an applicator, an applicator support fixedly attached to the applicator, a blade base configured for free rotation about the applicator and relative to the applicator support, the blade base defining an opening for the applicator, and one or more supports disposed on the blade base configured to releasably secure a blade to the blade base adjacent to the opening, wherein upon rotation of the blade base the blade forms an encircling groove in a cornea plug.

### CROSS-REFERENCE TO RELATED APPLICATION(S)

This application claims priority to U.S. Provisional Application No. 62/350,157 filed Jun. 8, 2022 and entitled “Glycerin-Preserved Corneal Plug, Mandrel for Fabricating Corneal Plugs and Associated Systems and Methods,” which is hereby incorporated by reference in its entirety under 35 U.S.C. § 119(e).

### TECHNICAL FIELD

The disclosure relates to devices, systems and methods for repairing corneal perforations via a corneal tissue plug and devices for the creation thereof.

### BACKGROUND

Corneal perforations can be caused by various factors such as trauma, infections, or surgical complications, and present a significant challenge to ophthalmologists. When definitive surgical repair is not possible in an operating room, the current standard of care for temporizing a corneal perforation is cyanoacrylate glue with or without a thin layer of reinforcing plastic fashioned ad hoc from a surgical drape. The strength of the perforation repair with cyanoacrylate glue derives from an adhesive bond between the glue and cornea; the patch is applied from outside of the eye onto the ocular surface. The intraocular pressure exerts an opposing force against the patch via aqueous fluid in the eye. The novelty of the disclosed implementations stem from, in part, the plug shape and its swelling properties.

The described systems, devices and methods overcome the limitations of the prior art by utilizing an optionally preserved corneal tissue plug that is configured to conform to the shape of the cornea and seal the perforation securely can be employed as an interim measure to stabilize corneal perforations and prevent further damage until definitive surgical repair can be performed. The temporary corneal perforation stabilizer described in this patent application allows for easy and efficient application, providing a protective barrier over the damaged cornea and promoting healing while maintaining the structural integrity of the eye.

### BRIEF SUMMARY

The disclosure relates to a corneal plug designed to seal corneal perforations and a device designed to fabricate the corneal plug.

In certain Examples, the disclosed technology relates to the creation of a plug with an encircling groove that is placed into holes or perforations in the cornea such that the groove interlocks with the edge of the perforation and the inner aspect of the plug makes a seal with the inner aspect of the cornea resulting from the intraocular pressure pushing the plug into place, which can then be reinforced with glue or its own intrinsic properties, such as swelling in response to moisture or other conditions. In further Examples, the technology includes certain devices and systems configured or otherwise constructed and arranged to create or otherwise cut the plug, and to further implementations are used to apply said device plug into the perforations. In certain Examples, the plug can be made of anything including various iterations of preserved cornea, non-preserved cornea, other human and non-human tissue preserved or not preserved, or biomaterial or a biomaterial/tissue hybrid

In Example 1, a corneal plug comprising preserved human corneal tissue with an encircling groove along a perimeter of the preserved human corneal tissue, wherein the encircling groove is configured for interlocking with edges of a corneal perforation.

Example 2 relates to the corneal plug of Example 1, wherein the corneal plug is self-retaining.

Example 3 relates to the corneal plug of any of Examples 1-2, wherein the corneal plug is reinforced in the corneal perforation with one or more of tissue adhesive, suture, and a central expansile shank.

Example 4 relates to the corneal plug of any of Examples 1-3, wherein the corneal plug is at least partially desiccating prior to implantation.

Example 5 relates to the corneal plug of any of Examples 1-4, wherein the encircling groove is about a 500 μm groove.

Example 6 relates to the corneal plug of any of Examples 1-5, wherein the encircling groove is a substantially v-shaped groove.

Example 7 relates to the corneal plug of any of Examples 1-6, further comprising a central partial thickness pocket for insertion of a sphere of desiccated cornea.

In Example 8, a method for creating a corneal plug comprising punching a plug from a corneal allograft that has been preserved, trimming the plug to a selected thickness with a cutting device, and cutting an encircling groove around the plug.

Example 9 relates to the method of Example 8, wherein the cutting device is selected from an ophthalmic crescent and a keratome blade.

Example 10 relates to the method of any of Examples 8-9, wherein a depth and profile of the encircling groove is selected to match a size and contour of a corneal perforation.

Example 11 relates to the method of any of Examples 8-10, further comprising saturating the plug with light sensitive material.

Example 12 relates to the method of any of Examples 8-11, further comprising adhering the plug to an applicator.

Example 13 relates to the method of any of Examples 8-12, further comprising forming a central partial thickness pocket in the plug.

In Example 14 a device for creating a corneal plug comprising an applicator, an applicator support fixedly attached to the applicator, a blade base configured for free rotation about the applicator and relative to the applicator support, the blade base defining an opening for the applicator, and one or more supports disposed on the blade base configured to releasably secure a blade to the blade base adjacent to the opening, wherein a cornea plug is held in the opening by the applicator and upon rotation of the blade base the blade forms an encircling groove in the cornea plug.

Example 15 relates to the device of Example 14, wherein the blade is selected from a #11 blade and a micrometer knife blade.

Example 16 relates to the device of any of Examples 14-15, wherein the one or more supports selected from walls and posts.

Example 17 relates to the device of any of Examples 14-16, further comprising a ramp on the blade base configured to orient the blade at an angle.

Example 18 relates to the device of any of Example 14-17, wherein the ramp is adjustable such that the angle of the blade is adjustable.

Example 19 relates to the device of any of Examples 14-18, wherein the blade base further comprises a plurality of geo-metrically disposed walls and ramps radiating from the opening, and wherein the plurality of geo-metrically disposed walls and ramps define a plurality of butting edges for a variety of cuts via a plurality of blades.

Example 20 relates to the device of any of Examples 14-19, further comprising suction tubing configured to convey suction to the opening and hold the cornea plug in place within the opening.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A shows a finished corneal plug made of glycerin preserved corneal tissue, according to one implementation.

FIG. 1B shows a corneal plug, no preserved in glycerin, according to one implementation.

FIG. 2A shows a manual preparation of a corneal plug, according to one implementation.

FIG. 2B shows a corneal plug that has been inserted into a perforation of a cornea, according to one implementation.

FIG. 3A shows a schematic representation of a cross section of a cornea where a plug is cut from the cornea graft, according to one implementation.

FIG. 3B shows a schematic representation of a plug being cut to desired thickness, according to one implementation.

FIG. 3C shows a schematic representation of a plug having an encircling groove formed therein, according to one implementation.

FIG. 4A is an external view of a preparation device, according to one implementation.

FIG. 4B is a cross-sectional view of a preparation device, according to one implementation.

FIG. 5A is a top, perspective view of a preparation device, according to one implementation.

FIG. 5B is a top, perspective view of a preparation device, according to one implementation.

FIG. 6A is a top, perspective view of a blade base, according to one implementation.

FIG. 6B is a top view of a blade base, according to one implementation.

FIG. 7A is a side view of an applicator with cornea plug at the end of the applicator and the cornea plug having an encircling groove, according to one implementation.

FIG. 7B is a cross-section view of punching a plug from a preserved cornea and affixing the plug to an applicator, according to one implementation.

FIG. 8A is a side view of a cornea plug having an encircling grooved, according to one implementation.

FIG. 8B is a side view of a cornea plug having an encircling grooved, according to one implementation.

### DETAILED DESCRIPTION

The various implementations described herein relate generally to a corneal plug designed to seal corneal perforations and a device designed to fabricate the corneal plug, shown generally in the figures at 1. In various implementations, the plug 1 is fabricated from a tissue material, which can be preserved or fresh corneal tissue derived from a cadaver or other source. In various implementations, the cornea tissue is preserved, such as in glycerin or other preservative that provides the desired modulus of elasticity and swelling properties that would be optimal for the plug 1 as understood by the skilled artisan. Fur

In various implementations, the disclosed system 10 utilizes glycerin-preserved human corneal tissue, which facilitates shaping of the tissue and promotes swelling of the plug 1 in the human recipient. However, it is readily-appreciated that non-human tissue, non-human tissue such as biosynthetic tissue or a hybrid thereof can be utilized in the formation of the plug, and that both preserved and non-preserved tissue can be used as described herein. And while certain of the described implementations make reference to the use of glycerin-preserved tissue being used, these other tissue or other material types are fully contemplated in each of the described examples. Certain non-limiting examples of other possible preservative or preparation techniques include, but are not limited to, alpha radiation, gamma radiation or other forms of applied radiation, alcohol, glycerin or other short-and long-term preservatives understood in the art.

In further implementations, the plug 1 has an encircling groove along its perimeter, which interlocks with the edges of a corneal perforation. The plug is self-retaining in the perforation but is reinforced with either tissue adhesive, suture, or a central expansile shank that simultaneously compresses the plug in the z-axis and makes it expand in the x-y axis.

Further implementations disclosed herein relate to certain devices which are configured to create the disclosed plugs via the rotation of either the blade or the plug itself.

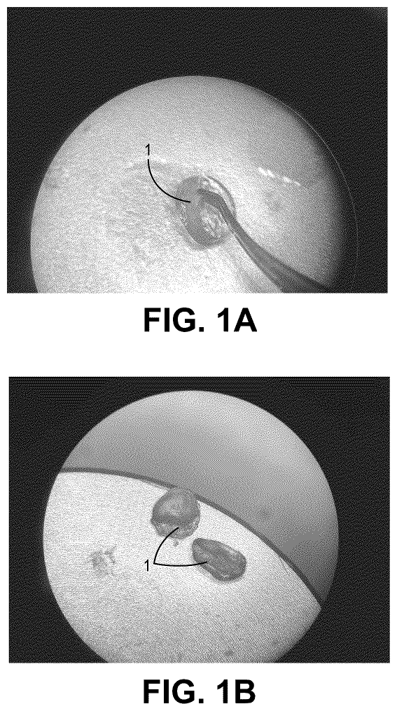

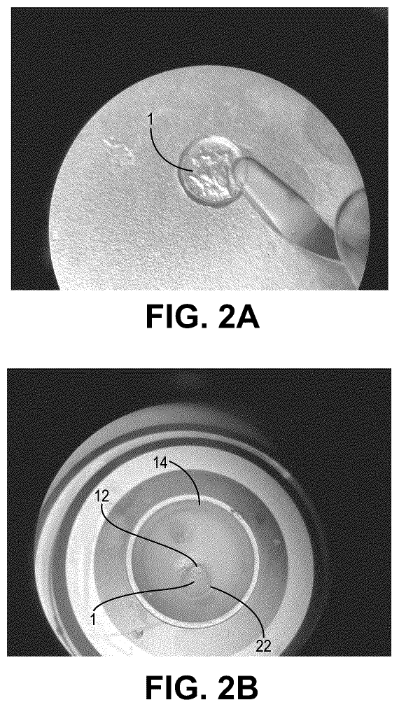

FIGS. 1A-2B show several views of plugs 1 under different circumstances. FIG. 1A shows a finished corneal plug 1 comprised of glycerin preserved corneal tissue. In contrast, FIG. 1B shows that non-preserved corneas can be difficult to fabricate into a plug. FIG. 2A shows a manual preparation of a plug 1. FIG. 2B shows a plug 1 self-retained in an ex vivo human simulated corneal surgery. That is, in FIG. 2B, a plug 1 has been inserted into the perforation 12 of the cornea 14.

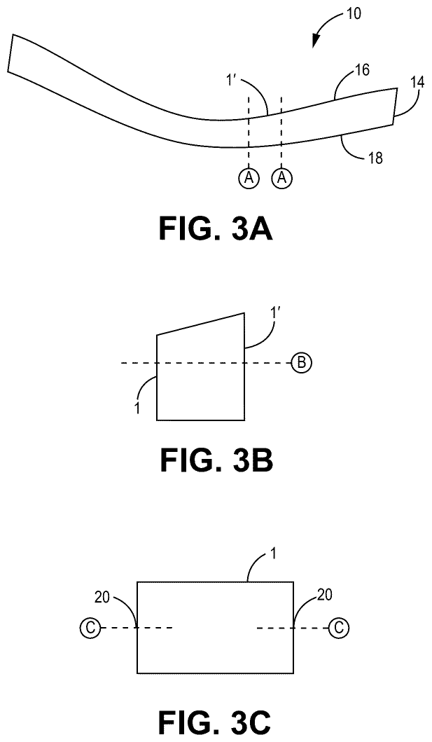

FIGS. 3A-3B depict the process of forming the plug 1 cutting process 10 according to certain implementations. The presently disclosed devices, systems and methods relate to the preparation of plugs 1 for use in such procedures. These implementations relate to both the methods described herein as well as the devices and systems used to achieve that process and result. These are generally referred to herein as a cutting process 10, but it is readily appreciated that such discussion contemplates various aspects of the disclosed technology. Generally, to fabricate a corneal plug, an eye bank technician renders a small corneal plug by punching it from either a full thickness corneal allograft or a partial thickness allograft, e.g. a 4 mm full-thickness plug, that has been preserved in glycerin. It is of course appreciated that other preservatives are of course possible.

In FIG. 3A, a cross-section of a cornea 14 is shown having an interior 16 and exterior 18. In this implementation, a round piece of tissue 1′ is cut from the cornea 14 using a cornea punch (shown at dashed lines A).

In FIG. 3B, the tissue 1′ is subsequently trimmed (shown at dashed line B) to the desired thickness via cutting device such as an ophthalmic crescent, keratome blade or similar device. Subsequently, the resulting plug 1 is cut in a ring-like fashion (shown with dashed lines C, in FIG. 3C) to introduce a ring 20 or groove 20 around the plug 1 in the result in a the finally prepared grooved plug 1.

It is understood that after the plug 1 has been placed in the patient cornea 14 perforation 12, the perforation's edges (shown in FIG. 2B at 22) act as the tongue that fits into the groove 20 of the plug 1, thereby rendering a circumferential tongue-in-groove interface between the corneal plug and the human recipient's perforated cornea. The encircling groove 20 can be of various depths and cross-sectional profiles, depending on the size and excavated contour of the corneal perforation. Further, since according to certain implementations the encircling groove 20 is fashioned into the corneal plug 1 after the tissue has been stored in glycerin—which in addition to preserving the tissue, also has the effect of desiccating it—the plug 1 is able to be rehydrated upon placement. Alternative methods of preservation can also have provide similar benefits, as would be understood.

That is, since the groove 20 is made in desiccated tissue, once the plug 1 is placed into the human recipient and it rehydrates, the dimensions of the plug 1 increase in width and height, thereby tightening the mechanical seal of the plug 1. In various implementations, the mechanical seal can optionally be further augmented by tissue adhesive, suture, or a central expansile shank that simultaneously compresses the plug in the z-axis and makes it expand in the x-y axis. In further implementations, photopolymerization is used to swell the plug 1 in place, as would be understood.

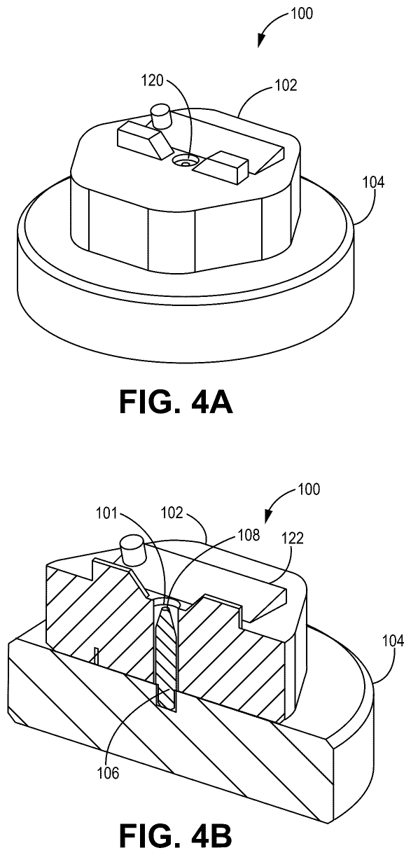

FIG. 4A depicts an external view of a preparation device 100 or cutting mechanism 100 according to certain implementations of the system 10, and FIG. 4B depicts a cross-sectional view of the device 100. The current state of the art for shaping corneal tissue is either freehand with a blade or with a femtosecond laser; historically, corneas have also been shaped with a cryolathe for refractive purposes. Freehand dissection is not consistent between replicates; a femtosecond laser is highly consistent but prohibitively expensive for this clinical indication; a cryolathe is also prohibitively expensive in addition to being better suited to shaping the surface contour of corneal tissue rather than cutting a groove in its profile. Glycerin or otherwise preserved corneas are much easier to mill than non-preserved corneas because the tissue is much more rigid and dense due to being desiccated by the chemical. The device 100 provides an inexpensive rotating platform to cut symmetric grooves 12 into the profile of corneal tissue that has been punched into small (e.g. 2-5 mm) plugs, as described above.

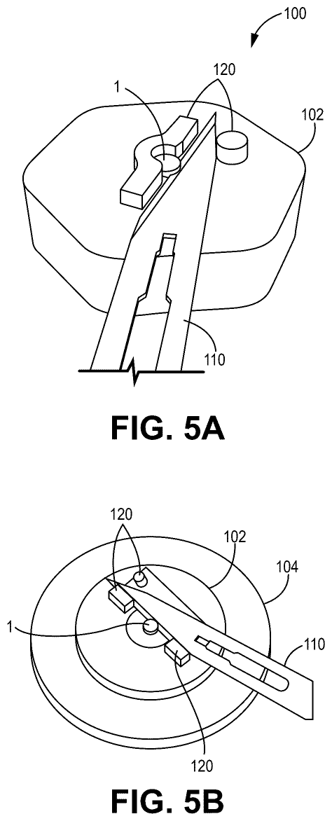

In the implementations of FIGS. 4A-5B, the device 100 has a blade base 102 and an applicator support 104, which is fixedly attached to the applicator 106 during use of the device. The blade base 102 and applicator support 104 are configured for free relative rotation in these implementations, however, such that the blade base 102 can freely rotate around the distal tip 108 of the applicator 106, as will be appreciated.

Continuing with FIGS. 4A-4B, and as shown further in FIGS. 5A-5B, the blade base 102 according to these implementations further comprises one or more supports 120 disposed atop the base 102 and configured to secure a blade 110, such as a #11 blade or other appropriate cutting device 110 in position adjacent to the opening 101 disposed within the base 102 that is configured to house the cornea plug 1 atop the distal tip 108 of the applicator 106 for the ring cut, as would be understood.

In various implementations, the optional support 120 or supports 120 can be walls, posts or other forms disposed on the top of the base 102 to assist the user in proper placement of the blade 110, as would be appreciated. In various implementations, an optional ramp 122 can also be disposed on the base 102 to further angle and support the blade during use of the device, including during rotation of the base 102 relative to the support 104. It is appreciated that in various implementations, the angle of the ramp 122 can vary depending on the needs and the optimal cut angle for the specific use case.

In one exemplary implementation, the plug 1 is mounted to the device 100 on the applicator 106 and a micrometer knife blade 110 is used to fashion or cut a groove 12 in the profile of the plug 1, e.g. about a 500 μm groove 12. It is understood that grooves 12 ranging from about 100 μm to about 1000 μm or more can be used, and that further implementations may feature grooves 12 outside that range.

In certain implementations, the groove 12 can be further shaped to have a v-pattern. The plug 1 is then stored in glycerin (optionally atop the applicator 106) until use at a tissue bank. In this example, a 4 mm plug with a central 3 mm plug would then be fit to a corneal perforation between 1-2.5 mm in diameter by a surgeon in a minor procedure room; if needed, the size of the perforation could be enlarged to accommodate the plug. Once in place, the plug 1 can be glued, sutured, or compressed/expanded via a central shank.

In various implementations, the mechanism of cutting the lamellar ring or groove 12 cut of the plug 1 may be modified. The mechanism of cutting the height of the plug may be modified. Cutting with a blade or blade-like device may be replaced with laser cutting in certain implementations. The shape of the corneal plug 1 may be modified such that one side is wider in diameter than the other side, but a ring lamellar cut or groove 12 is still present in the narrower aspect of the plug.

The corneal plug 1 may be modified to augment its natural swelling properties; this modification may be biochemical, mechanical, or both. The plug 1 may be modified to have a central partial thickness pocket into which a sphere of desiccated cornea is pushed; the sphere then swells, thereby making the plug swell in the horizontal axis. The plug 1 itself may be saturated with a light-sensitive material that causes the plug to swell. The plug 1 may be modified to make it function as a depot for medications. The plug 1 may be further secured with an adhesive such as cyanoacrylate glue or a light-sensitive material that polymerizes under light. Elements of the device 100 and the fabrication system may be used for other surgical purposes in and outside of ophthalmology.

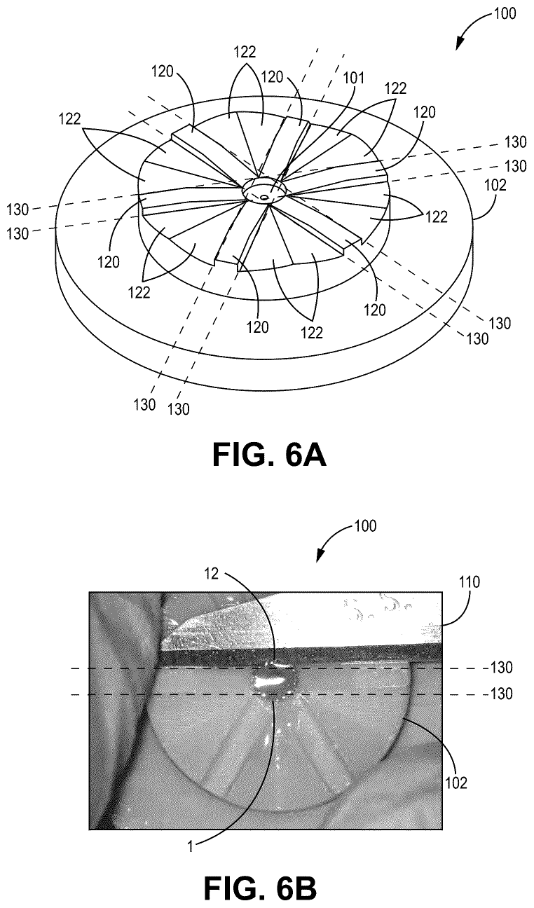

FIG. 6A depicts a further implementation of a blade base 102 which in certain implementations can be disposed atop the applicator support 104 as previously described. This base comprises a plurality of geometrically-disposed walls 120 and ramps 122 radiating from the plug opening 101. In these implementations, the plurality of walls 120 and ramps 122 define a plurality of cutting edges 130 disposed atop the blade base 102 configured to allow for the performance of a plurality of cuts via the blade 110, as is shown for example in FIG. 6B. It is understood that in these implementations, cutting is performed by rotating the blade base 102 to cut through each of the plurality of cutting edges 130, thereby forming the groove 12 around the plug 1.

In certain of these implementations, the base 102 is in fluidic communication with suction, such that suction is pulled through the bottom of the blue bases. In one such exemplary implementation, a piece of IV tubing is run under the base 102 and exits out of the side of the applicator support 104. This tubing is in turn connected to a syringe that can pull suction once the plug 1 is placed into the opening 101, as would be understood. In these implementations, pulling suction ensures for a reduction in movement of the cornea plug 1 while performing the groove cuts, which can score the cut into the side of the plug 1 more accurately since the tissue is normally flexible.

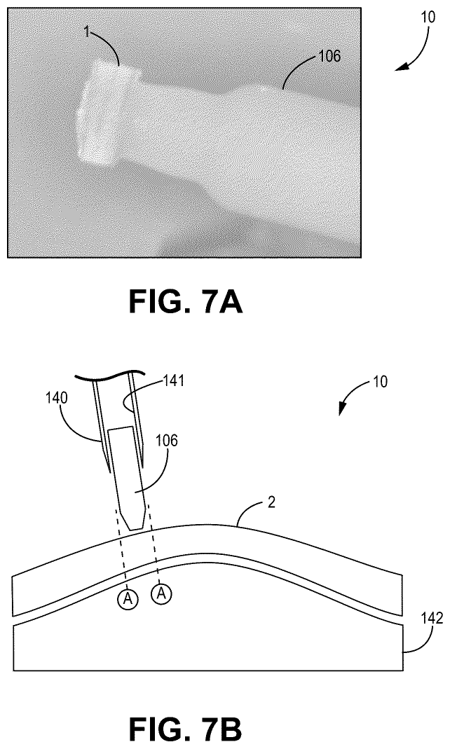

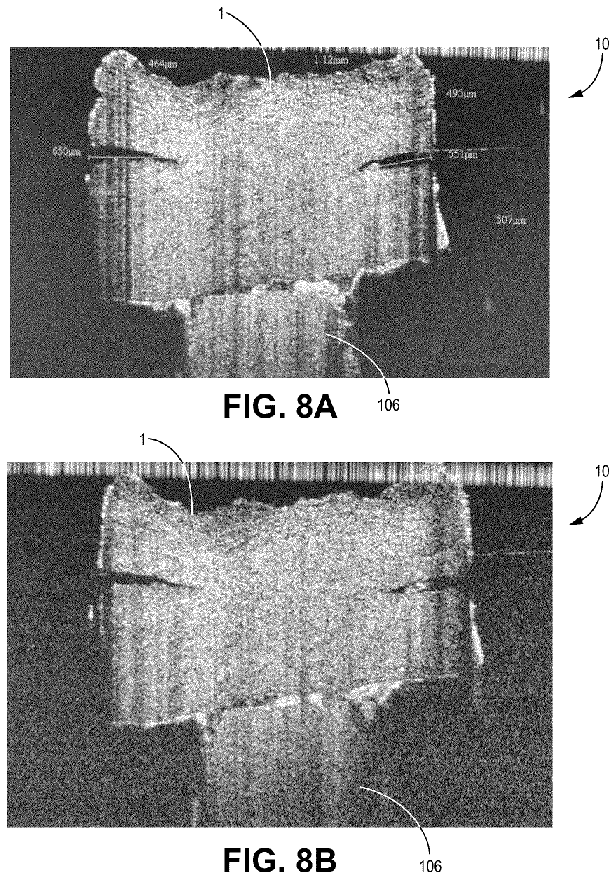

FIG. 7A depicts an image of an exemplary implementation of an applicator 106 having the plug 1 with the groove 12 disposed circumferentially about the outer surface. It is understood that in certain implementations, the plug 1 can be affixed to the applicator 106 directly from the punch of the cornea, as is shown in FIG. 7B. In FIG. 7B, the applicator 106 is disposed within a hollow punch 140 having an internal lumen 141 with a defined internal diameter. In these implementations, it is appreciated that the punch 140 is in slidable communication with the applicator 106 such that it can be extended past the distal tip 108 of the applicator 106 to punch the plug 1, as shown at dashed lines A. In various of these implementations, an adhesive such as cyanoacrylate glue is applied to the cornea 2 prior to the punch, and in certain of these implementations the cornea 2 can be dried beforehand to ensure better adhesion between applicator and plug during a simultaneous punch and adhesion approach under these implementations. Further, in these implementations an optional base 142 that is configured to model the curvature of a cornea in vivo. FIGS. 8A-8B depict optical coherence tomography (OCT) scans of plugs 1 affixed to applicators 106 according to certain implementations.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Although the disclosure has been described with reference to preferred embodiments, persons skilled in the art will recognize that changes may be made in form and detail without departing from the spirit and scope of the disclosed apparatus, systems and methods.

What is claimed is:

1. A corneal plug comprising: preserved human corneal tissue with an encircling groove along a perimeter of the preserved human corneal tissue, wherein the encircling groove is configured for interlocking with edges of a corneal perforation.

2. The corneal plug of claim 1, wherein the corneal plug is self-retaining.

3. The corneal plug of claim 2, wherein the corneal plug is reinforced in the corneal perforation with one or more of tissue adhesive, suture, and a central expansile shank.

4. The corneal plug of claim 1, wherein the corneal plug is at least partially desiccating prior to implantation.

5. The corneal plug of claim 1, wherein the encircling groove is a 500 μm groove.

6. The corneal plug of claim 1, wherein the encircling groove is a substantially v-shaped groove.

7. The corneal plug of claim 1, further comprising a central partial thickness pocket for insertion of a sphere of desiccated cornea.

8. A method for creating a corneal plug comprising:
punching a plug from a corneal allograft that has been preserved;
trimming the plug to a selected thickness with a cutting device; and
cutting an encircling groove around the plug.

9. The method of claim 8, wherein the cutting device is selected from an ophthalmic crescent and a keratome blade.

10. The method of claim 8, wherein a depth and profile of the encircling groove is selected to match a size and contour of a corneal perforation.

11. The method of claim 8, further comprising saturating the plug with light sensitive material.

12. The method of claim 8, further comprising adhering the plug to an applicator.

13. The method of claim 8, further comprising forming a central partial thickness pocket in the plug.

14. A device for creating a corneal plug comprising:
(a) an applicator;
(b) an applicator support fixedly attached to the applicator;
(c) a blade base configured for free rotation about the applicator and relative to the applicator support, the blade base defining an opening for the applicator; and
(d) one or more supports disposed on the blade base configured to releasably secure a blade to the blade base adjacent to the opening,
wherein a cornea plug is configured to be held in the opening by the applicator and upon rotation of the blade base the blade forms an encircling groove in the cornea plug.

15. The device of claim 14, wherein the blade is selected from a #11 blade and a micrometer knife blade.

16. The device of claim 14, wherein the one or more supports selected from walls and posts.

17. The device of claim 14, further comprising a ramp on the blade base configured to orient the blade at an angle.

18. The device of claim 17, wherein the ramp is adjustable such that the angle of the blade is adjustable.

19. The device of claim 14, wherein the blade base further comprises a plurality of geo-metrically disposed walls and ramps radiating from the opening, and wherein the plurality of geo-metrically disposed walls and ramps define a plurality of butting edges for a variety of cuts via a plurality of blades.

20. The device of claim 14, further comprising suction tubing configured to convey suction to the opening and hold the cornea plug in place within the opening.
